(19) United States
(12) Patent Application Publication
(10) Pub. No.: US 2015/0065798 A1
(43) Pub. Date: Mar. 5, 2015

(54) ELECTRONIC ENDOSCOPE DEVICE AND IMAGING MODULE THEREFOR

(71) Applicant: FUJIFILM Corporation, Tokyo (JP)

(72) Inventor: Osamu KURODA, Ashigarakami-gun (JP)

(73) Assignee: FUJIFILM Corporation, Tokyo (JP)

(21) Appl. No.: 14/532,107

(22) Filed: Nov. 4, 2014

Related U.S. Application Data
(63) Continuation of application No. PCT/JP2013/061843, filed on Apr. 23, 2013.

(30) Foreign Application Priority Data
May 28, 2012 (JP) 2012-120646

(51) Int. Cl. A61B 1/05; A61B 1/00

(52) U.S. Cl. CPC A61B 1/05; A61B 1/00186
USPC 600/109

(57) ABSTRACT

Provided are an imaging module for an endoscope that favorably blocks incidence of flare light to an imaging element and an electronic endoscope device including the same. The imaging module for an endoscope includes an objective lens optical system; a transparent optical member 56; an imaging element 58 that is of a microlens non-mounting type; an adhesive layer; and a light shielding mask 121 for a measure against flare that is interposed between a light emission surface 56a and a light incidence surface, is formed with an opening 121a, which matches an image circle that passes through the objective lens optical system and the transparent optical member 56 and is formed on the light incidence surface of the imaging element 58 and has a smaller diameter than the image circle, and is provided immediately before the light incidence surface of the imaging element 58.

### CROSS-REFERENCE TO RELATED APPLICATIONS

This application is a Continuation of PCT International Application No. PCT/JP2013/061843 filed on Apr. 23, 2013, which claims priority under 35 U.S.C. §119(a) to Japanese Patent Application No. 2012-120646 filed May 28, 2012. Each of the above application is hereby expressly incorporated by reference, in its entirety, into the present application.

### BACKGROUND OF THE INVENTION

1. Field of the Invention

The present invention relates to an electronic endoscope device and an imaging module therefor, and particularly, to an electronic endoscope device and an imaging module therefor in which measures against flare are taken.

2. Description of the Related Art

In an electronic endoscope device in which an imaging module is built in an endoscope tip portion and an observation image of the inside of a body cavity of a subject imaged by this imaging module is displayed on a monitoring screen, measures against flare are taken in order to improve the quality of a captured image.

For example, in an electronic endoscope device described in JP2009-288682A, a flare diaphragm is provided at a stage before a prism, which changes a light path of incident light substantially at a right angle on a light receiving surface side of an imaging element, such that flare light reflected within the incident light path does not enter the imaging element.

Additionally, in the electronic endoscope device given in JP1997-205590A (JP-H09-205590A), a flare diaphragm is provided at a peripheral edge portion of a cover glass, which protects a light receiving surface of an imaging element, such that flare light reflected within the incident light path does not enter the imaging element.

A flare diaphragm is formed by a light shielding mask or a light shielding film. Generally, the light shielding film used with the imaging element is provided to block light that enters pixels (photodiodes) of an optical black (OB) portion in order to detect black level, and is provided inside the imaging element. In contrast, the light shielding film (light shielding mask) for a flare diaphragm is provided at a stage before the imaging element outside the imaging element. Although both of the light shielding films have the same light shielding function, one light shielding film is provided for the purpose of completely blocking the incident light to the black level detection pixels, and the other light shielding film is provided for the purpose of suppressing incidence of flare light of the incident light into the pixels. For this reason, a position or a region where the light shielding film is provided varies.

Although the flare diaphragm is provided at the stage before the imaging element, it is better to provide the flare diaphragm at a position as close to the imaging element as possible. Since the flare diaphragm described in JP2009-288682A is provided at the stage before the prism arranged immediately before the imaging element, the incidence of the flare light generated between the flare diaphragm and the light receiving surface of the imaging element to the imaging element cannot be blocked.

The flare diaphragm described in JP1997-205590A (JP-H09-205590A) is provided on the back side (imaging element side) of the cover glass attached on insulating resin, which covers connecting terminals or wirebonding provided around the light receiving surface of the imaging element, so as to cover the light receiving surface of the imaging element. For this reason, the back surface of the cover glass provided with the flare diaphragm and the light receiving surface of the imaging element may be separated from each other, and the flare light reflected by an inner peripheral surface of a resin layer may enter the light receiving surface of the imaging element.

### SUMMARY OF THE INVENTION

An object of the invention is to provide an imaging module for an endoscope that can favorably remove a flare, and an electronic endoscope device that houses the imaging module in an endoscope tip portion.

An imaging module for an endoscope of the invention includes an objective lens optical system; a transparent optical member that takes in subject image light from the objective lens optical system and emits the image light from a planar light emission surface; a photoelectric conversion layer laminated type imaging element which is of a microlens non-mounting type and has a planar light incidence surface; an adhesive layer that bonds together the light emission surface of the transparent optical member and the light incidence surface of the imaging element; and a light shielding mask for a measure against flare that is interposed between the light emission surface and the light incidence surface and is formed with an opening, which matches an image circle that passes through the objective lens optical system and the transparent optical member and is formed on the light incidence surface of the imaging element and which has a smaller diameter than the image circle. The light shielding mask is provided immediately before the light incidence surface of the imaging element.

The electronic endoscope device of the invention has the above imaging module for an endoscope built in an endoscope tip portion.

According to the invention, since the light shielding mask for a measure against flare is provided immediately before the light incidence surface of the imaging element, it is possible to shield incidence of flare light to the imaging element, and it is possible to capture a high-quality image.

### BRIEF DESCRIPTION OF THE DRAWINGS

FIG. 1 is an overall configuration view of an electronic endoscope device related to an embodiment of the invention.

FIG. 2 is a front view of a tip surface of a tip portion of the electronic endoscope device illustrated in FIG. 1.

FIG. 3 is a longitudinal cross-sectional view of the tip portion of the electronic endoscope device illustrated in FIG. 1.

FIG. 4 is an enlarged cross-sectional schematic view of an imaging element portion of FIG. 3.

FIG. 5 is an exploded perspective view of a prism, a flare diaphragm, and an imaging element of the embodiment illustrated in FIG. 3.

FIG. 6 is a view illustrating an example of the relationship between an image circle and the imaging element.

### DESCRIPTION OF THE PREFERRED EMBODIMENTS

Hereinafter, an embodiment of the invention will be described with reference to the drawings.

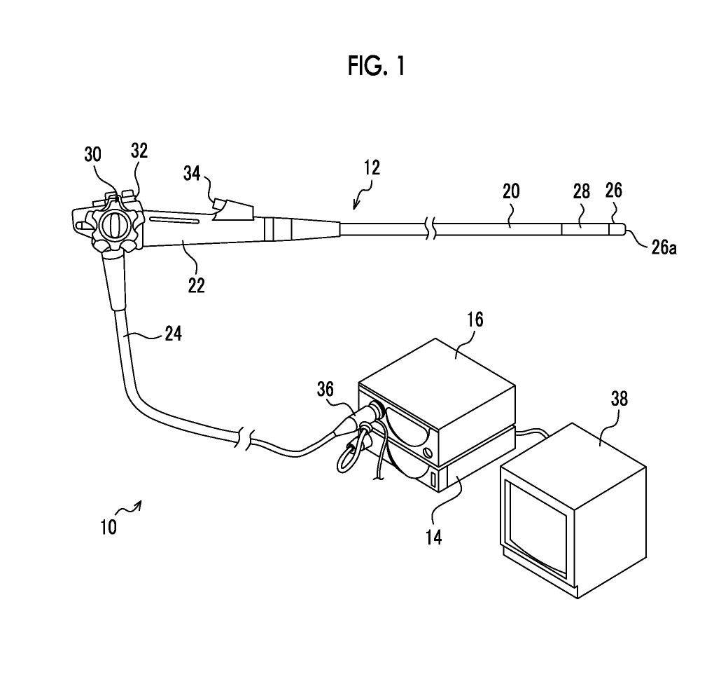

FIG. 1 is a configuration view illustrating an overall system of an electronic endoscope device related to an embodiment of the invention.

The electronic endoscope device (endoscope system) 10 of the present embodiment is constituted by an endoscope 12, and a processor unit 14 and a light source unit 16 that constitute a body device. The endoscope 12 includes a flexible insertion section 20 that is inserted into a patient's (subject's) body cavity, an operation section 22 provided continuously with a base end portion of the insertion section 20, and a universal cord 24 connected to the processor unit 14 and the light source unit 16.

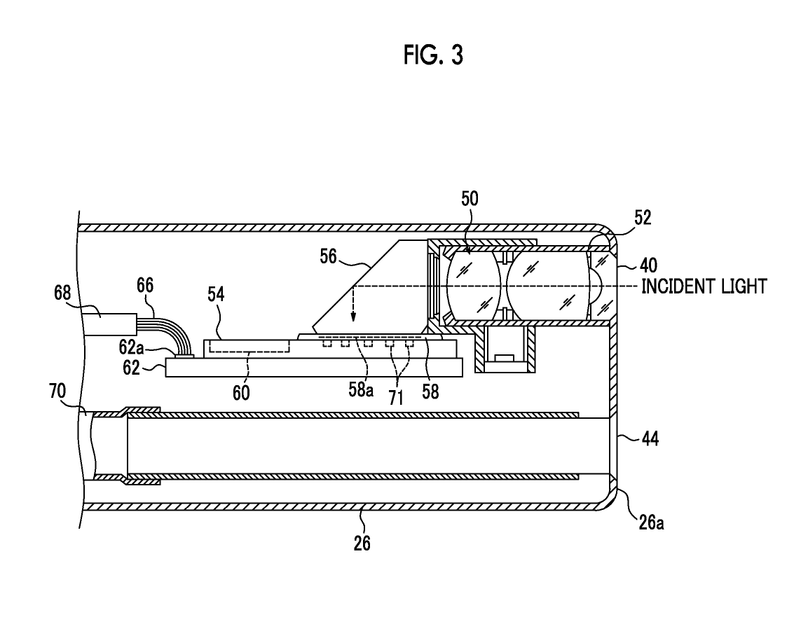

A tip portion 26 is provided continuously with a tip of the insertion section 20, and an imaging chip 54 (refer to FIG. 3) for picking up an image of the inside of body cavity (imaging device) is built within the tip portion 26. A bending portion 28 formed by coupling a plurality of bending pieces together is provided behind the tip portion 26. When an angle knob 30 provided at the operation section 22 is operated, a wire inserted into the insertion section 20 is pushed/pulled and the bending portion 28 makes bending motions in vertical and horizontal directions. Accordingly, the tip portion 26 is directed towards a desired direction within the body cavity.

A connector 36 is provided at a base end of the universal cord 24. The connector 36 is a composite type connector, and is not only connected to the processor unit 14 and but also connected to the light source unit 16.

The processor unit 14 supplies electric power to the endoscope 12 via a cable 68 (refer to FIG. 3) inserted through the universal cord 24 to control the driving of the imaging chip 54, receives imaging signals transmitted via the cable 68 from the imaging chip 54, and performs various signal processing on the received imaging signals to convert the imaging signals into image data.

The image data converted by the processor unit 14 is displayed on a monitor 38 cable-connected to the processor unit 14 as an endoscope pick-up image (observation image). Additionally, the processor unit 14 is also electrically connected to the light source unit 16 via the connector 36, and generally controls the operation of the electronic endoscope device 10 including the light source unit 16.

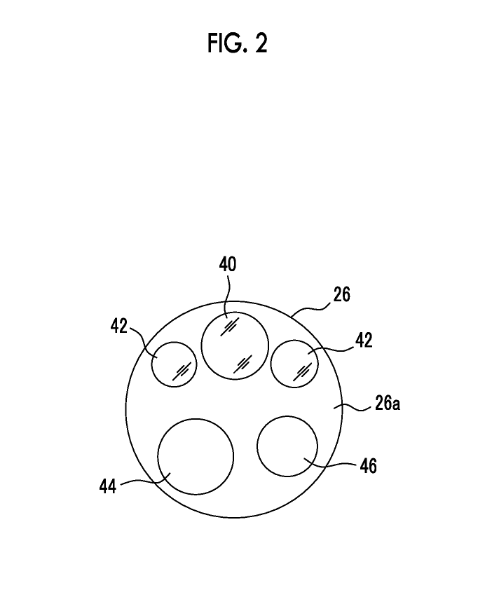

FIG. 2 is a front view illustrating a tip surface 26a of the tip portion 26 of the endoscope 12. As illustrated in FIG. 2, the tip surface 26a of the tip portion 26 is provided with an observation window 40, illumination windows 42, a forceps outlet 44, and an air/water supply nozzle 46.

The observation window 40 is arranged so as to be eccentric from the center and one side of the tip surface 26a. Two illumination windows 42 are disposed at positions symmetrical with respect to the observation window 40, and irradiate a part to be observed within the body cavity from the light source unit 16.

The forceps outlet 44 is connected to a forceps channel 70 (refer to FIG. 3) disposed within the insertion section 20, and communicates with a forceps port 34 (refer to FIG. 1) provided in the operation section 22. Various treatment tools having an injection needle, a high-frequency knife, and the like disposed at tips thereof are inserted through the forceps port 34, and the tips of the various treatment tools are passed into the body cavity from the forceps outlet 44.

The air/water supply nozzle 46 jets washing water and air supplied from an air/water supply unit built in the light source unit 16 toward the observation window 40 or the body cavity in response to the operation of an air/water supply button 32 (refer to FIG. 1) provided at the operation section 22.

FIG. 3 is a longitudinal cross-sectional view of the tip portion 26 of the endoscope 12. As illustrated in FIG. 3, a lens barrel 52 that holds an objective lens optical system 50 for taking in image light of the part to be observed within the body cavity is disposed in the depths of the observation window 40. The lens barrel 52 which is attached so that an optical axis of the objective lens optical system 50 becomes parallel to a central axis of the insertion section 20. A prism 56 that bends the image light of the part to be observed, which has went through the objective lens optical system 50, substantially at a right angle and guides the bent image light towards the imaging chip 54 is connected to a rear end of the lens barrel 52.

The imaging chip 54 is an imaging chip in which a single plate type imaging element 58 for capturing a color image and a signal read-out circuit are formed on a semiconductor chip, and a photoelectric conversion layer as an organic layer is laminated on the signal read-out circuit, in which a peripheral circuit 60 that performs the driving of the imaging element 58 and the input/output of a signal is formed on the semiconductor chip, and is mounted on a supporting substrate 62.

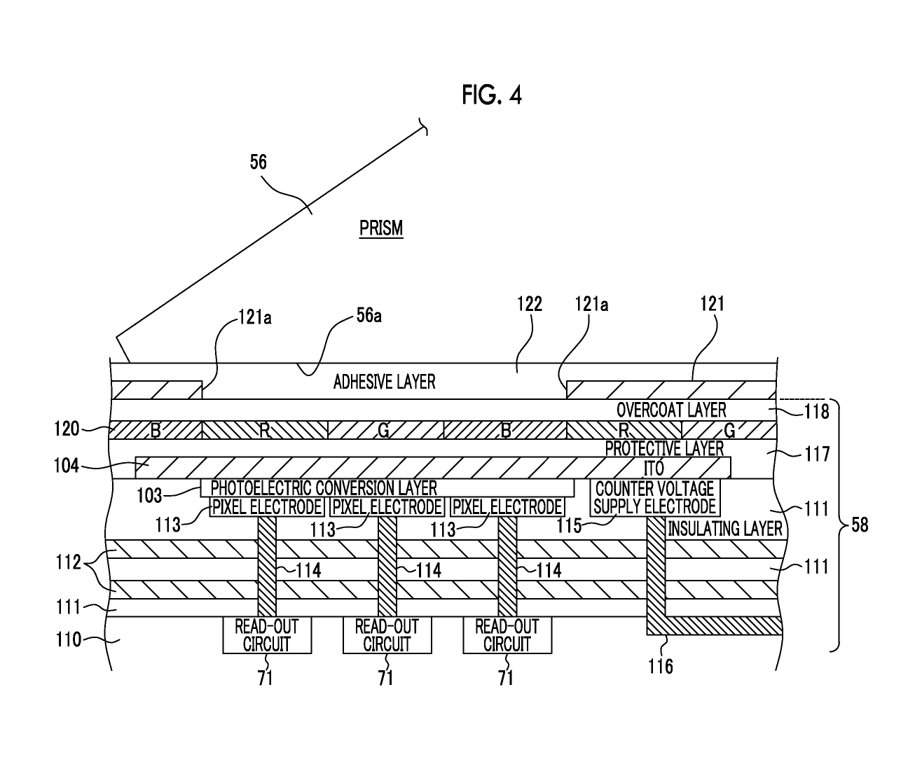

An imaging surface (light receiving surface) 58a of the imaging element 58 is arranged so as to face a light emission surface of the prism 56. As will be described below with reference to FIG. 4 for details, the light receiving surface of the imaging element 58 is bonded to the light emission surface of the prism 56 via the flare diaphragm with an adhesive.

A plurality of input/output terminals 62a are provided side by side in a width direction of the supporting substrate 62 at a rear end of the supporting substrate 62 provided to extend toward a rear end of the insertion section 20. Signal lines 66 for intermediating exchange of various signals with the processor unit 14 via the universal cord 24 are joined to the input/output terminals 62a. The input/output terminals 62a are electrically connected to the peripheral circuit 60 within the imaging chip 54 via wiring lines, bonding pads (not shown), and the like that are formed on the supporting substrate 62.

The signal lines 66 are collectively inserted into a flexible tubular cable 68. The cable 68 is inserted through the insertion section 20, the operation section 22, and the universal cord 24, respectively, and is connected to the connector 36.

Additionally, although illustration is omitted in FIGS. 2 and 3, an illumination unit is provided in the depths of the illumination unit 42. A light emission end of a light guide that guides illumination light from the light source unit 16 is disposed at the illumination unit 42, and this light emission end is provided so as to face the illumination unit 42. Similar to the cable 68, the light guide is inserted through the insertion section 20, the operation section 22, and the universal cord 24, respectively, and an incident end is connected to the connector 36.

FIG. 4 is an enlarged cross-sectional schematic view of a portion of the imaging element 58 illustrated in FIG. 3. A photoelectric conversion layer laminated type imaging element 58 is formed on a semiconductor substrate 110. MOS circuits 71, such as CMOS circuits as signal read-out circuits, are formed for each pixel on a surface portion of the semiconductor substrate 110. The signal read-out circuits are of CCD types. The photoelectric conversion layer laminated type imaging element is described in JP2011-243945A or the like that was previously filed by the applicant of the present invention and was already disclosed.

An insulating layer 111 is laminated on the surface of the semiconductor substrate 110, and a wiring line layer 112 is buried in the insulating layer 111. The wiring line layer 112 also plays a function of a shielding plate in such a manner that incident light transmitted and leaked through an upper layer does not enter the signal read-out circuits 71 or the like.

A plurality of pixel electrode films 113 divided for each pixel and arranged in a square lattice when viewed from above are formed on the surface of the insulating layer 111. A vertical wiring line 114 that reaches even the surface of the semiconductor substrate 110 is erected from each pixel electrode film 113, and each vertical wiring line 114 is connected to a signal charge storage unit (illustration is omitted) formed on the surface of the semiconductor substrate 110.

The signal read-out circuit 71 provided for each pixel is adapted to read out a signal according to the amount of signal charges stored in a corresponding signal charge storage unit to the outside as a subject image signal. In addition, in the present embodiment, the pixel electrode films 113 are provided in an effective pixel region and OB portions.

One sheet of a light receiving layer 103 having a photoelectric conversion function is laminated common to the respective pixel electrode films on the plurality of pixel electrode films 113 arrayed and formed in a square lattice. Similarly, one sheet of an upper electrode film (also referred to as a counter electrode film and a common electrode layer) 104 is laminated as an upper layer on a light incidence side with respect to the pixel electrode films 113 on the light receiving layer 103. The light receiving surface 58a described in FIG. 3 corresponds to the light receiving layer 103, and a photoelectric conversion unit is formed by the light receiving layer 103, the lower electrode layers (pixel electrode films) 113 that vertically sandwich the light receiving layer, and the upper electrode film 104.

The upper electrode film 104 is in a state where the upper electrode film is electrically connected to a counter voltage supply electrode layer 115 exposed to the surface of the insulating layer 111 via a wiring line 116, and a desired voltage is applied from the outside of the imaging element via the wiring line 116 to the upper electrode film.

A protective layer 117 is laminated on the upper electrode film 104, and color filters 120 corresponding to the pixel electrode films 113, respectively, are laminated on the protective layer. For example, color filters in red (R) green (G) blue (B) that are the three primary colors are Bayer-arrayed, or color filters for a complementary color system are laminated. An overcoat layer (protective layer) 118 is laminated on the color filters 120.

The above-described upper electrode film 104 is made of conductive materials that are transparent with respect to incident light in order to make light enter the light receiving layer 103. As a material for the upper electrode film 104, transparent conducting oxide (TCO) with a higher transmittance with respect to visible light and a small resistance value can be used.

Although a thin film of metal, such as Au (gold), can also be used as the upper electrode film, the resistance value thereof increases extremely if the thickness of the film is made small in order to obtain a transmittance of 90% or more. Therefore, TCO is more preferable. As TCO, particularly, indium tin oxide (ITO), indium oxide, tin oxide, fluorine-doped tin oxide (FTO), zinc oxide, aluminum-dope zinc oxide (AZO), titanium oxide, or the like can be preferably used. ITO is the most preferable from the viewpoints of process simplicity, low resistance, and transparency. In addition, although the upper electrode film 104 is constituted by one sheet common to all the pixels in the embodiment, a configuration may be adopted in which an upper electrode film is divided for each pixel and each divided film is connected to a power source.

The lower electrode layers (pixel electrode films) 113 are thin films divided for each pixel, and are made of transparent or opaque conductive materials. As the materials for the lower electrode layers 113, metals, such as Cr, In, Al, Ag, W, and TiN (titanium nitride), and TCO can be used.

The protective layer 117 and the overcoat layer 118 are made of transparent insulating materials, a silicon oxide film, a silicon nitride film, zirconium oxide, tantalum oxide, titanium oxide, hafnium oxide, magnesium oxide, alumina (Al₂O₃), polyparaxylene-based resin, acrylate resin, perfluorinated transparent resin (Cytop), or the like.

The protective layer 117 and the overcoat layer 118 may be a multilayer film that is formed by well-known techniques, such as a chemical vapor deposition method (CVD method) and an atomic layer deposition method (ALD ALCVD) and is combined with a plurality of insulating films that are deposited by the CVD, the atomic layer deposition method, or the like if necessary. A smoothing layer and an overcoat layer are smoothened and flattened by removing convex portions through chemical-mechanical polishing (CMP) after films are formed.

The protective layer 117 and the overcoat layer 118 are desirably as thin as possible within the thickness so as to keep there function respectively and are preferably 0.1 μm to 10 μm in thickness, respectively.

As described with reference to FIG. 3, the peripheral circuit 60 is also formed on the semiconductor substrate 110. Although the imaging element 58 is from the semiconductor substrate 110 to the overcoat layer 118, the surface of the overcoat layer 118 of the imaging element 58 is directly bonded to the light emission surface 56a of the prism 56 with an adhesive layer 122.

In this case, for example, a flare diaphragm (light shielding mask) 121 in which a opening 121a with a predetermined diameter is opened in a black film (for example, 10 μm to 30 μm in thickness) without reflection, such as a graphite film, is provided immediately before the overcoat layer 118 that is a light incidence surface of the imaging element 58, and is sandwiched between the overcoat layer and the adhesive layer 122. The adhesive layer 122 flatten the thickness of the light shielding mask 121 by filling up a gap between the light emission surface 56a of the prism 56 and the surface of the overcoat layer 118, and anchors the light emission surface 56a of the prism 56 and the surface of the overcoat layer 118 so that these surfaces becomes parallel to each other.

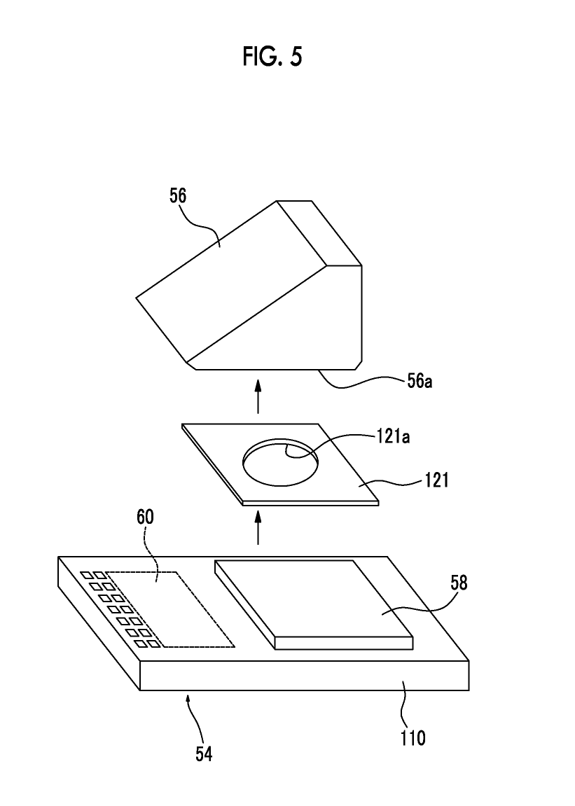

FIG. 5 is an exploded perspective view of the imaging chip 54, the light shielding mask (flare diaphragm) 121, and the prism 56. The illustration of the objective lens optical system 50 to which the prism 56 is attached is omitted. An imaging module is manufactured by bonding the surface of the imaging element 58 of the imaging chip 54 to the light emission surface of the prism 56 via the light shielding mask 121 with an adhesive.

The opening 121a opened in the light shielding mask 121 opens so as to be concentrically matched with an image circle formed in the light receiving surface of the imaging element 58 via the objective lens optical system 50 and the prism 56 and so as to become a circle with a slightly smaller diameter than this image circle.

The imaging element 58 of the present embodiment is of a top lens (microlens) non-mounting type. This is because, even if the top lens is not provided, the whole light receiving surface can be used as a light receiving element (photoelectric conversion unit), and the light receiving sensitivity is high.

For this reason, the surface (surface of the overcoat layer 118) of the imaging element 58 is planar. This surface is brought into close contact with and bonded to the light emission surface (plane) of the prism 56 with the adhesive layer 122 with the light shielding mask (flare diaphragm) 121 sandwiched therebewteen. The regions that are brought into close contact with each other are the whole surfaces of regions where the light emission surface of the prism 56 and the surface of the imaging element 58 overlap each other.

As a result, the prism 56 and the imaging element 58 are anchored to each other without a slight gap between the both, and the surface of the imaging element 58 is protected by the prism 56, and moisture prevention is also achieved.

If the adhesive layer 122 is applied so as to fill a gap between the top lenses when respective pixels of an imaging element are mounted with the top lenses, the refractive index differences between the top lenses and the gap become small, and the function of the lenses degrades. For this reason, in the case of the top lens mounted type of the imaging element, as described JP2010-98066A, it is necessary to bond a cover glass slightly apart from a top lens so as not to impair the lens function.

That is, it is necessary to provide the flare diaphragm 121 in a gap between the top lens and the cover glass. In such a case, there is a concern that stray light generated as incident light may be irregularly reflected after passing through the flare diaphragm 121 enters each pixel, and a flare image may be captured. Additionally, since a gap is present, it is necessary to achieve moisture prevention.

However, in the embodiment of the above-described invention, the above gap is not present. Therefore, a flare can be suppressed to the minimum, and moisture prevention of the light receiving surface of the imaging element does not need to be separately achieved.

In addition, in the above-described embodiment, the flare diaphragm 121 is formed of the black film. However, a black coating material may be thickly applied to the surface of the overcoat layer 118 by a printing technique to form the flare diaphragm 121, and the overcoat layer 118 of the imaging element 58 may quickly be bonded to the light emission surface of the prism 56 with the adhesive layer 122.

Otherwise, contrary to this, a black coating material may be thickly applied to the light emission surface of the prism 56 by the printing technique to form the flare diaphragm 121, and the overcoat layer 118 of the imaging element 58 may quickly be bonded to the light emission surface of the prism 56 with the adhesive layer 122.

As the adhesive layer 122, for example, thermosetting transparent resin or ultraviolet-curable transparent resin may be used. The adhesive layer 122 can be cured by applying heat or ultraviolet rays to an adhesive surface while the prism 56 and the imaging element 58 are maintained in a close contact state.

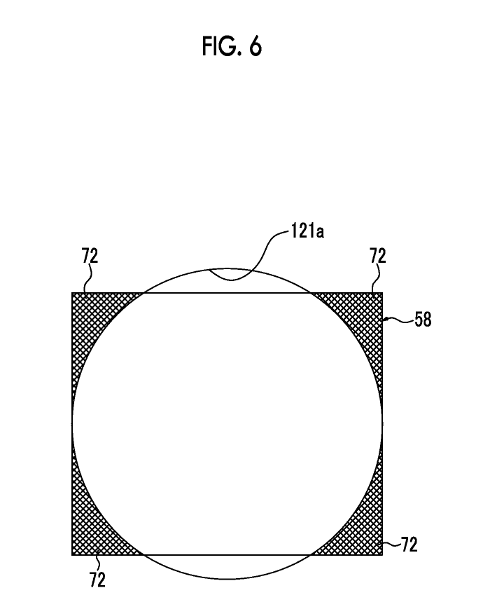

FIG. 6 is a view of the imaging element 58 side viewed from the light emission surface of the prism 56, and is a view illustrating the relationship between the image circle (light shielding mask opening 121a) and the imaging element 58. The light receiving surface of the rectangular imaging element 58 is formed with an image circle of incident light that passes through the objective lens optical system and is bent in a right-angled direction by the prism 56, and is provided with the light shielding mask (flare diaphragm) 121 in which the opening 121a that matches this image circle is opened.

In the illustrated example, regions light-shielded by the flare diaphragm 121 are formed at four corners (cross-hatched regions) 72 of the rectangular light receiving surface of the imaging element 58. In the present embodiment, the above-described pixel electrode films 113, light receiving layer 103, upper electrode film 104, and color filters 120 are also provided in the regions 72.

That is, the OB portions are provided in the regions 72 light-shielded by the flare diaphragm 121, and the flare diaphragm 121 is also made to serve as light shielding films of the OB portions. Accordingly, it becomes unnecessary to provide the OB portions separately from the region of the imaging element 58, and miniaturization of the imaging element 58 can be achieved (for example, in the photoelectric conversion layer laminated type imaging element described in the above-described JP2011-243945, the imaging element and the OB portions are provided in separate regions).

In addition, miniaturization may be achieved not by providing the regions 72 with the OB portions but by other circuits (for example, the peripheral circuit).

By providing the regions 72 with the OB portions and mounting the color filters on the OB pixels, it is possible to obtain OB signals of R filter mounted pixels, OB signals of G filter mounted pixels, and OB signals of B filter mounted pixels. The offset amount of a black level signal varies for each color of the mounted color filters in many cases. For this reason, it is possible to obtain the OB pixels integrated value for every color of the color filters and accurately obtain the offset amount, and it is possible to capture a high-quality image with little noise.

It is also possible not to read detection signals of the OB pixels other than when needed, but to achieve a high-speed signal read-out and power-savings.

Similar to the above-described embodiment, the reason why the light incidence surface of the imaging element 58 can be brought into close contact with the light emission surface 56a of the prism 56 throughout an overlapping region is because of the structure in which the photoelectric conversion layer (light receiving layer) 103 or the like is laminated on the semiconductor substrate 110. That is, the reason is because the photoelectric conversion layer laminated portion has the structure (structure in which wirebonding does not become obstructive) in which the laminated portion protrudes upward from the surface of the semiconductor substrate. Since connection pads to be connected to circuit elements formed on a semiconductor is formed on the surface portion of the semiconductor substrate and the light incidence surface of the imaging element is formed at an upper position apart from the semiconductor substrate, the light incidence surface of the imaging element can be brought into close contact with and bonded to the light emission surface of the prism 56.

For this reason, the above-described embodiment is also applicable to the signal read-out circuit and its connecting terminal, and a backside irradiation type imaging element in which the incidence plane of incident light is located on the opposite side. The backside irradiation type imaging element also does not need to provide the top lenses, and a light incidence surface thereof is a plane. For this reason, a light incidence surface of the imaging element can be brought into close contact with and fixed to a wide region of the light emission surface of the prism.

Additionally, in the above-described embodiment, the optical axis of incident light is bent in the right-angled direction using the prism 56. However, for example, as described in JP1997-205590A (JP-H09-205590A), in the case of the imaging element that receives the image light emitted from the objective lens optical system perpendicularly as it is at the light receiving surface, a transparent cover glass of a parallel flat plate may be used instead of the prism without using the prism. Even in this case, the flare diaphragm 121 may be interposed between the cover glass and the light receiving surface of the imaging element, both of which are brought into close contact with and bonded to each other.

As described above, an imaging module for an endoscope of the embodiment includes an objective lens optical system; a transparent optical member that takes in subject image light from the objective lens optical system and emits the image light from a planar light emission surface; an imaging element which is of a microlens non-mounting type and has a planar light incidence surface; an adhesive layer that bonds together the light emission surface of the transparent optical member and the light incidence surface of the imaging element; and a light shielding mask for a measure against flare that is interposed between the light emission surface and the light incidence surface, is formed with an opening, which matches an image circle that passes through the objective lens optical system and the transparent optical member and is formed on the light incidence surface of the imaging element, and has a smaller diameter than the image circle. The light shielding mask is provided immediately before the light incidence surface of the imaging element.

Additionally, in the imaging module for an endoscope of the embodiment, the imaging element is of a photoelectric conversion layer laminated type.

Additionally, in the imaging module for an endoscope of the embodiment, the transparent optical member is a right-angle prism that bends a light path of the subject image light emitted from the objective lens optical system in a substantially right-angled direction and makes the image light enter the light incidence surface of the imaging element.

Additionally, in the imaging module for an endoscope of the embodiment, the transparent optical member is a cover glass made of a parallel plate that transmits the subject image light emitted from the objective lens optical system and makes the image light enter the light incidence surface of the imaging element.

Additionally, in the imaging module for an endoscope of the invention, the imaging element is a single plate type imaging element for capturing a color image in which color filters for the three primary colors or a complementary color system are laminated on each pixel.

Additionally, in the imaging module for an endoscope of the embodiment, an OB pixel for black level detection is provided in a region of the imaging element, which is light-shielded by the light shielding mask for a measure against flare, and the light shielding mask for a measure against flare is also made to serve as a light shielding film of the OB pixel.

Additionally, in the imaging module for an endoscope of the embodiment, color filters for detecting a black level signal for each color of the color filters are laminated on the OB pixel.

Additionally, in the imaging module for an endoscope of the embodiment, a peripheral circuit of the imaging element is formed in a region of the imaging element, which is light-shielded by the light shielding mask for a measure against flare.

Additionally, the electronic endoscope device of the embodiment has the imaging module for an endoscope described in any one of the above built in an endoscope tip portion.

According to the embodiment described above, since the light shielding mask for a measure against flare is provided immediately before the light incidence surface of the imaging element, it is possible to prevent incidence of flare light to the light receiving surface of the imaging element, and a high-quality image can be captured.

Since the imaging module for an endoscope related to the invention can favorably prevent incidence of flare light to the light receiving surface of the imaging element, this imaging module is useful if the module is built in the scope tip portion of the electronic endoscope device.

What is claimed is:

1. An imaging module for an endoscope comprising:
an objective lens optical system;
a transparent optical member that takes in subject image light from the objective lens optical system and emits the image light from a planar light emission surface;
a photoelectric conversion layer laminated type imaging element which is of a microlens non-mounting type and has a planar light incidence surface;
an adhesive layer that bonds together the light emission surface of the transparent optical member and the light incidence surface of the imaging element; and
a light shielding mask that is interposed between the light emission surface and the light incidence surface, is formed with an opening, which matches an image circle that passes through the objective lens optical system and the transparent optical member and is formed on the light incidence surface of the imaging element, and has a smaller diameter than the image circle,
wherein the light shielding mask is provided immediately before the light incidence surface of the imaging element.

2. The imaging module for an endoscope according to claim 1,
wherein the transparent optical member is a right-angle prism that bends a light path of the subject image light emitted from the objective lens optical system in a substantially right-angled direction and makes the image light enter the light incidence surface of the imaging element.

3. The imaging module for an endoscope according to claim 1,
wherein the transparent optical member is a cover glass made of a parallel plate that transmits the subject image light emitted from the objective lens optical system and makes the image light enter the light incidence surface of the imaging element.

4. The imaging module for an endoscope according to claim 1,
wherein the imaging element is a single plate type imaging element for capturing a color image in which color filters for the three primary colors or a complementary color system are laminated on each pixel.

5. The imaging module for an endoscope according to claim 2,
wherein the imaging element is a single plate type imaging element for capturing a color image in which color filters for the three primary colors or a complementary color system are laminated on each pixel.

6. The imaging module for an endoscope according to claim 3,
wherein the imaging element is a single plate type imaging element for capturing a color image in which color filters for the three primary colors or a complementary color system are laminated on each pixel.

7. The imaging module for an endoscope according to claim 4,
wherein an OB pixel for black level detection is provided in a region of the imaging element, which is light-shielded by the light shielding mask, and the light shielding mask is also made to serve as a light shielding film of the OB pixel.

8. The imaging module for an endoscope according to claim 5,
wherein an OB pixel for black level detection is provided in a region of the imaging element, which is light-shielded by the light shielding mask, and the light shielding mask is also made to serve as a light shielding film of the OB pixel.

9. The imaging module for an endoscope according to claim 6,
wherein an OB pixel for black level detection is provided in a region of the imaging element, which is light-shielded by the light shielding mask, and the light shielding mask is also made to serve as a light shielding film of the OB pixel.

10. The imaging module for an endoscope according to claim 7,
wherein color filters for detecting a black level signal for each color of the color filters are laminated on the OB pixel.

11. The imaging module for an endoscope according to claim 8,
wherein color filters for detecting a black level signal for each color of the color filters are laminated on the OB pixel.

12. The imaging module for an endoscope according to claim 9,
wherein color filters for detecting a black level signal for each color of the color filters are laminated on the OB pixel.

13. The imaging module for an endoscope according to claim 4,
wherein a peripheral circuit of the imaging element is formed in a region of the imaging element, which is light-shielded by the light shielding mask.

14. The imaging module for an endoscope according to claim 5,
wherein a peripheral circuit of the imaging element is formed in a region of the imaging element, which is light-shielded by the light shielding mask.

15. The imaging module for an endoscope according to claim 6,
wherein a peripheral circuit of the imaging element is formed in a region of the imaging element, which is light-shielded by the light shielding mask.

16. An electronic endoscope device having the imaging module for an endoscope according to claim 1 built in an endoscope tip portion.

17. An electronic endoscope device having the imaging module for an endoscope according to claim 2 built in an endoscope tip portion.

18. An electronic endoscope device having the imaging module for an endoscope according to claim 3 built in an endoscope tip portion.

19. An electronic endoscope device having the imaging module for an endoscope according to claim 4 built in an endoscope tip portion.

20. An electronic endoscope device having the imaging module for an endoscope according to claim 7 built in an endoscope tip portion.
